(19) United States
(12) Patent Application Publication
(10) Pub. No.: US 2010/0074931 A1
(43) Pub. Date: Mar. 25, 2010

(54) USE OF A HIGHLY EMULSIFIED SILICONE OIL (HES) FOR THE PRODUCTION OF A MEDICAMENT

(76) Inventor: Heinrich Exner, Moeckern (DE)

Correspondence Address: PATENT CENTRAL LLC;Stephan A. Pendorf, 1401 Hollywood Boulevard, Hollywood, FL, 33020, US

(21) Appl. No.: 12/526,540

(22) PCT Filed: Feb. 4, 2008
(86) PCT No.: PCT/DE2008/000226

§ 371 Date: Aug. 10, 2009

(30) Foreign Application Priority Data
Feb. 9, 2007 (DE) 10 2007 007 698.5

(51) Int. Cl. A61K 9/107; A61K 31/80; A61K 9/70

(52) U.S. Cl. 424/402; 424/78.37

(57) ABSTRACT

The invention relates to the use of a silicone oil emulsion comprising 0.01-85 wt. % of silicones, particularly polydimethylsiloxanes, 0.01-90 wt. % of hydrophobic emulsifier that has an HLB value ranging from 1 to 7 and/or a hydrophilic emulsifier that has an HLB value ranging from 7 to 14 and/or 0.01-90 wt. % of a mixture of one or more hydrophobic and hydrophilic emulsifiers having an HLB value ranging from 1 to 14, and 0.1-99 wt. % of biocompatible saline solution, for producing a medicament as such or in combination with other medicaments or carrier materials for treating septic infections triggered by bacterial, viral, parasitic, and fungal causative agents or diseases in animals and humans caused by traumas.

The invention concerns the use of a highly emulsified silicone oil-emulsion for production of a medicament.

Bacterial infections are treated as a rule with anti-bacterial effective medicaments such as, for example, antibiotics, which bring about that the infection causing bacteria are in large part killed off and the organism is placed into condition to eliminate the remaining bacteria and to recover.

As a rule, antibiotics are well tolerated and have a broad therapeutic range. The main side effects are allergies, interference with the intestinal flora (antibiotic-associated diarrhea and the occurrence of fungal infections, rarely pseudomembraneous colitis). Rarely the antibiotics cause organ toxic effects, for example kidney damage and hearing loss caused by Gentamicin. Several antibiotics such as Bacitracin or Colistin exhibit in systemic (internal) administration such strong side effects that they can only be applied locally. These are referred to as local antibiotics. In certain infections, such as for example Lues, antibiotics can trigger a so-called Herxheimer reaction, in which the organism is overwhelmed with toxins from the killed bacteria.

The basic problem, which is associated with the use of antibiotics, is the danger of development of resistance against the general antibiotic, whereby this becomes ineffective against the infection-causing bacteria and in consequence becomes non effective. In such cases, the doctor is forced to change the medication and to administer a different antibiotic, with the always-present danger that the organism develops new resistance.

It is thus desirable to develop medicaments which exhibit antibiotic-like or antibacterial effect or rather make it possible to forgo antibiotics, and with which the immune system is brought into condition to combat the pathogens on its own, without however leading to resistance.

In medicine and cosmetics silicone oils are known as components of skin protective salves, salve bases, for stabilizing hairstyles, as fixatives for fragrances, and components of toothpaste. For example in WO 02/38160 salves and lotions for cosmetic treatment of the skin are described, which contain dimethicone.

In EP 1 704 853 A2 a topically applicable composition developed purely for cosmetic purposes for reduction of wrinkles, dark circles around the eyes, etc. is disclosed, which is based on a silicone oil emulsion and additionally contains retinoides and copper containing peptides.

Silicone oils are dielectric and dipolar. In most cases a dielectric fluid obtains its characteristics by polarization. When a dielectric fluid is introduced into an electrical field, the dipoles contained in the dielectric fluid orient according to the field. The term dipole generally refers to the presence of two opposing charges—e.g., a molecule with an electrical positive and an electrical negative end. Due to this polarization the dielectric fluid is under tension and stores energy, which can be given off again as soon as the electrical field has been removed.

The larger the surface area to which the dipolar silicone molecules have been applied, the greater also the charge acceptance. It follows that silicone oil emulsions are suitable for electron transfers due to their large surface area. The terminal boundaries of silicone oil emulsions due to decomposition or, as the case may be, metabolism of its components is of great significance with respect to its clinical effects. In rat paw tests the transport of the silicone oils out of the emulsion could already be observed beginning of the first day following the application of the emulsion. After the fourth day the transport by monocytes and neutrophilic granulocytes already substantially decreased.

Reactive oxidizing intermediates include, e.g., super oxide anions, hydrogen peroxide as well as hydroxyl radicals. Oxygen is essential in organisms for energy production. Besides energy production, unsaturated oxides are also produced due to UV-radiation, heat, immune reactions such as phagocytosis, mechanical and chemical irritants and all work performed by the organism, which it must produce for maintenance of its integrity. The dangerous reactive oxygen species (ROS) or reactive oxygen intermediates (ROI), which are produced in various side reactions, are deactivated in aerobic organisms by diverse protective mechanisms. The super oxide radical is the most toxic ROS. It is produced in inflammation reactions by activated macrophages. By means of a superoxide dismutase this radical is converted into the likewise toxic hydrogen peroxide, and this is destroyed by catalysis and subsequently by various peroxidases, which occur in mammals as well as in plants.

Oxidative stress occurs when antioxidative (oxidation inhibiting) processes decrease in comparison to oxidative processes and the balance of equilibrium of the two processes is shifted in the human organism towards that of oxidation. This process can develop into a chain reaction. Oxygen radicals trigger a central signal transduction cascade in cells. This so-called mitogenic activating phosphate kinase (MAP) triggers a cascade, which can transition into proliferative or apoptotic effects. An elevated mRNA-expression MAP dependent gene was also discovered. Mitogenes are substances which excite mitosis and therewith the proliferation of cells, and at the same time the transformation from stem cells.

As long as the organisms maintain the balance between antioxidative and oxidative processes via their redox systems, no damage occurs in the cell systems. The ageing of the organism has been traced to the influence of, besides the genotype, recently also of these oxidative processes on the DNA structures of the cells.

The reduction in the concentration of reactive oxygen intermediates in metabolism of animals and humans leads, among other things, in the case of septic infections to a rapid and clear healing or reduction in the patient-stressing symptoms.

For the treatment of redox systems of organisms that have fallen into imbalance, it has been known to treat animal and humans in experiments with antioxidants such as antioxidative effective chemical compounds such as vitamins, selenium compounds among other things. An influencing of disease processes therewith was however until know not, or hardly, been observable.

The physico-chemical characteristics of silicone oil emulsions allow a completely new field for the treatment of disease processes to be opened, wherein the course of the disease can be positively influenced by the reduction of unsaturated oxygen intermediates. The pathologic effects occurring in the disease process due to unsaturated oxygen intermediates and the consequences can be buffered electrostatically early-on and impart to the course of the disease a substantially improved prognosis. The negative charges of the unsaturated oxygen intermediates are buffered by the silicone oil emulsion. The neutralization of this charge occurs via the body itself.

The dipole character of a non- or only slightly-emulsified silicone oil has little significance in the elimination of pathogenic oxygen compounds due to its low surface area.

The invention is thus concerned with the task of proposing a highly emulsified silicone oil emulsion for topical, parenteral, in particular intramuscular and/or subcutaneous application, which is free of proteins and which can be utilized alone or in combination with other topical, parenteral, in particular intramuscular and/or subcutaneous applicable effective substances for production of a medicament for treatment of septic infections.

This task is accomplished with a highly emulsified silicone oil emulsion with the composition:
0.01-85 w/w % silicone, particularly polydimethylsiloxanes,
0.01-90 w/w % hydrophobic emulsifier with a HLB value of 1 to 7 and/or a hydrophilic emulsifier with a HLB value of 7 to 14 and/or 0.01-90 w/w % of a mixture of one or more hydrophobic and hydrophilic emulsifiers with a HLB value of 1 to 14,
0.1-99 w/w % biocompatible saline solutions
   for producing a medicament for treatment of septic infections caused by bacterial, viral and fungal pathogens or by trauma caused disease in animals and humans.

A septic infection or also sepsis is an infectious disease not associated with one specific pathogen, but rather can occur as a consequence of an infection with various bacteria, virus, fungi or protozoa. Sepsis is based on a failure of the defensive mechanisms of the organism. Pathogenic basis is the invasion of pathogens or the result of their toxins in the circulatory system. The result of this process depends on three factors:
number, pathogenicity and virulence of the pathogen
condition of the organismic defense mechanisms
reaction of the host organism

On the part of the host organism, currently an initial reaction of monocytes and macrophages is believed to occur. As a consequence there is a release and activity of cytokines (Tumor-Necrosis-Factor (TNF), interleukins, and interferons) and lipid mediators (thromboxane, prostaglandins, leukotrines and platelet agglutinating factor (PAF)). The direct cause for the circulation destabilization is among other things interference with clotting, inhibition of catecholamine sensitive receptors, endothelial cell damage and an excessive NO— and OH— ion release.

Systemic inflammatory response syndrome (SIRS) is the medical term for body defensive inflammatory defensive reaction of the total organism. The cause for SIRS could be: immunological, chemical reasons (for example acute pancreatitis), shock, burns, major operations (above all operations with cardio-pulmonary bypass such as major heart operations or involving large wound surfaces), severe trauma, or various major diseases (for example necrotizing pancreatitis).

Two of the following criteria must be satisfied in order to make the diagnosis of SIRS:
body temperature >38° C. or <36° C.
heart pulse rate >90/min
tachypnea: breathing frequency >20/min or hyperventilation with pCO₂ <4.3 kPa (33 mmHg)
leukocytosis (>12000/μl) or leucopenia (<4000/μl) or shifting to the left (that is >10% immature leukocytes formed in differential blood composition).

Following a diagnosis of SIRS and after testing positive for infection one refers to sepsis. The treatment of sepsis with the inventive highly emulsified silicone oil emulsion is based on the following physico-chemical principals: In the inventive highly emulsified silicone oil emulsion the silicone oils are highly emulsified to form a so-called HES (high emulsified silicone oil) and exhibit a very large surface area. The dipolar solvents and the emulsifiers employed on a basis of polyoxyethylene present in the highly emulsified silicone oil emulsion besides the silicone oil increase the dipole character of the highly emulsified silicone oil emulsion. With the HES the same effect is achieved that the organism reaches by means of the enzyme super oxide dismutase, catalysis and peroxidases, namely the deactivation of the ROS and ROI. As a consequence the pathological effects and the downstream products occurring due to the unsaturated oxygen intermediates in the disease process can be electrostatically buffered early-on and the healing process can be positively influenced.

It was determined that the highly emulsified silicone oil emulsion in the inventive composition following topical, parenteral, in particular intramuscular and/or subcutaneous application surprisingly exhibits a good effectiveness in the above listed indications. The highly emulsified silicone oil emulsion is prepared according to the type of application. It can be solid, semi-solid or even liquid. For this emulsion, aides can be included, which make possible the desired generic preparation. Such aides can be selected from the group “cocoa butter, solidified plant oils, mixtures of mono-, di- and tri-glyceride saturated fatty acids, waxes”. As additional aides it can be incorporated besides cetyl stearyl alcohol, beeswax, glycerin monostearate, aluminum stearate, bentonite, celluloses, liquid triglycerides, paraffins, highly dispersed silica or diatomaceous earth, calcium carbonate, magnesium oxide, and antioxidants of the inventive highly emulsified silicone oil emulsion. By the application of this highly emulsified silicone oil emulsion an enduring and strong effect is achieved.

The highly emulsified silicone oil emulsion unburdens the detoxification system and serves in the stimulation of the general defensive mechanism of the immune system in response to diverse diseases, in particular septic infections or bacterial diseases. The dosing is preferably between 50 μl to 100 ml depending upon the constitution of the patient. This dosing sufficed, according to the present observations, in order to trigger a targeted treatment and to stimulate the immune system.

It has further been observed that the combination of the inventive highly emulsified silicone oil emulsion with other topic or parenteral applicable substances lead to a surprising effect. If the inventive highly emulsified silicone oil emulsion is combined with at least one substance selected from the active substance group antibiotic, corticosteroids, antiparkinson agents, agents against multiple sclerosis, cytostatic drugs, neuro- and psychopharmaceuticals, immune stimulants and analgesics, then the amount of medication with these active substances, in comparison to the conventional medication without being combined with the inventive highly emulsified silicone oil emulsion, can be reduced by up to 50% and in certain cases by more than 60%. In this manner, full or enhanced effectiveness of these active substances can be achieved while the side effects can be reduced to a minimum.

The highly emulsified silicone oil emulsion is produced with the composition
0.01-85 w/w % silicone, in particular polydimethylsiloxanes,
0.01-90 w/w % hydrophobic emulsifier with a HLB-level of 1 to 7 and/or a hydrophilic emulsifier with a HLB-level of 7 to 14 and/or 0.01-90 w/w % of a mixture of one or more hydrophobic and hydrophilic emulsifiers with a HLB-level of 1 to 14, and
0.1-99 w/w % biocompatible saline solution.

Preferably the proportion of a hydrophobic emulsifier with a HLB-level of 1 to 7 and/or a hydrophilic emulsifier with a HLB-level of 7 to 14 comprises 0.01-35 w/w % and/or a mixture of one or more hydrophobic and hydrophilic emulsifiers with a HLB-level of 1 to 14 comprises 0.1-35 w/w %.

The highly emulsified silicone oil emulsion can be an oil-in-water, water-in-oil, as well as a double emulsion.

The highly emulsified silicone oil emulsion can additionally contain one of the components 0.0001-1.0 w/w % chelate former and glycerol in a concentration of 0.01-50 w/w %.

The emulsifier can be comprised of 25-35 w/w % sorbitan trioleate, 25-35 w/w % cetyl stearyl alcohol and 35-45 w/w % polysorbate 80.

A highly emulsified silicone oil emulsion based on polydimethylsiloxane, which has a degree of polymerization of n=20-1000 and a kinetic viscosity with 20-2000 mm²/s, has been found to be particularly effective in accordance with the invention.

The silicone oil emulsion can contain EDTA Na/Ca salt in a percentage by weight of 0.0001-0.1 g/ml.

The highly emulsified silicone oil emulsion can be applied upon carrier materials, preferably selected from the group “cork, solid paraffins, waxes, resins, woven textiles.”

The components of a highly emulsified silicone oil emulsion are mixed in accordance with known emulsification chemical principals. The highly emulsified silicone oil emulsion is filtered under sterile conditions, autoclaviable and can be frozen. If the highly emulsified silicone oil emulsion separates due to one of these measures, it can subsequently be re-emulsified.

It has been found that the inventive highly emulsified silicone oil emulsion is suitable for achieving particularly good effects in the case of use as medicament for treatment of one of sepsis or SIRS, triggered by a microbial initiated infection or trauma. This type of infections of the human or animal incurred by legionella, staphylococcus, klebsiella, haemophilus influenza, rickettsia (spotted fever), mycobacteria, mycoplasmids, ureaplasmids, neisseria (meningitis, Waterhouse-Friderichsen-syndrome, gonorrhea), pseudomonades, bordetella (pertussis), corynebacterium (diphtheriae), chlamydia can be treated with the inventive highly emulsified silicone oil emulsion. Likewise, the use of the highly emulsified silicone oil emulsion is indicated in infections of the digestive tract by campylobacter (diarrhea), Escherichia coli, Escherichia proteus, salmonella, shigella, yersinia, vibrio, enterococci, clostriadium, listeria. Further treatable infections include borreliae—(borreliosis), treponema pallidum—(syphilis), brucella—, francisella-(tulerensis) and leptospirosis infections.

Sepsis producing treatable viral infections include: influenza (true flu), common cold, cough (due to rhinovirus, RSV, para-influenza virus), measles, mumps, rubella (German measles), fifth disease (erythema infectiosum, due to parvovirus B19), three-day-fever (exanthema subitum, caused by HHV 6), chicken pox (caused by Varizella-Zoster-virus), mononucleosis (caused by Eppstein-Barr-virus), diarrhea (dysentery, caused by rotovirus, adenovirus), hepatitis A or E (jaundice), infantile paralysis (polio), herpes labialis, FSME (caused by tick bite), RSSE (caused by tick bite), herpes genitalis, herpes simplex, measles, fifth disease, rubella, cytomegalovirus infection, AIDS (by HIV), hepatitis B (jaundice), herpes.

The medicament can be employed in humans as well as in animals and can be prepared for topical and parenteral application.

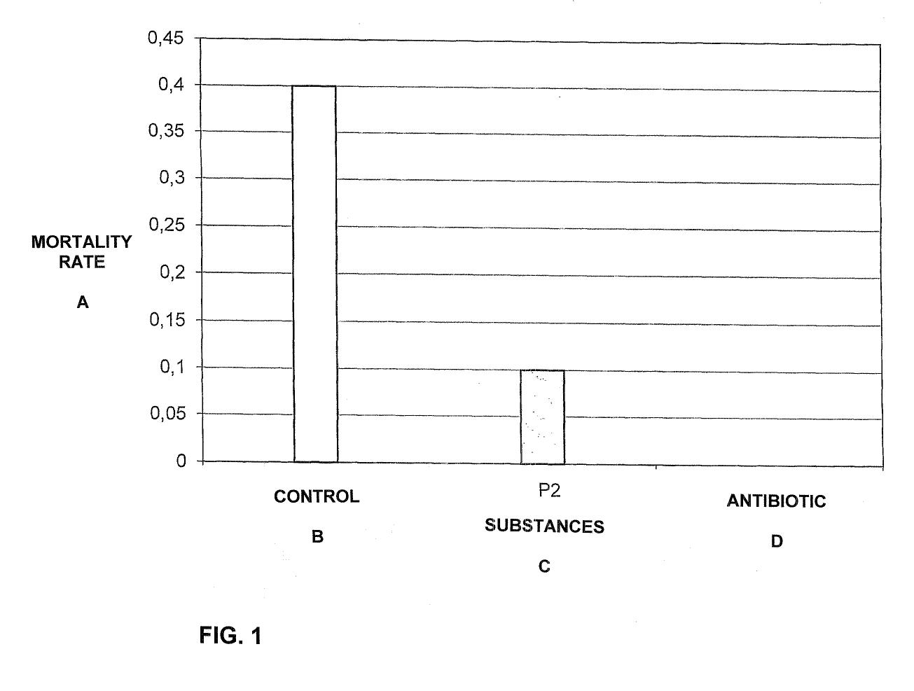

In the following the inventive highly emulsified silicone oil emulsion is described in greater detail on the basis of FIG. 1:

FIG. 1 shows the result of a treatment of sepsis in CH3-mice. The sepsis was induced by a peritoneal infection of sensitive CH3-mice with the microorganism Streptococcus pyogenes A20 (human M-type 23-isolate) with an infection dose of 1×10⁴ cfu in 200 ul PBS per mouse. In each group 10 infected mice were employed. The mortality rate after 3 days is shown in FIG. 1. The control group remained untreated following infection period. Group P2 was treated with the inventive highly emulsified silicone oil emulsion, wherein 50 μl of the medicament was injected. The antibiotic group received an antibiotic following infection.

As a result, by the treatment of the sepsis with the purely highly emulsified silicone oil emulsion the mortality rate was reduced by 75%.

1-11. (canceled)

12. A highly emulsified silicone oil emulsion based medicament for the treatment of SIRS and/or sepsis in mammals, comprising
0.01-85 w/w % silicone,
0.01-90 w/w % of at least one emulsifier selected from
hydrophobic emulsifier with a HLB-level of 1 to 7,
hydrophilic emulsifier with a HLB-level of 7 to 14, and
mixture of one or more hydrophobic and hydrophilic emulsifiers with a HLB-level of 1 to 14, and
0.1-99 w/w % biocompatible saline solution.

13. The highly emulsified silicone oil emulsion based medicament according to claim 12, wherein the silicone is a polydimethylsiloxane, and wherein the polydimethylsiloxane is emulsified.

14. The highly emulsified silicone oil emulsion based medicament according to claim 13, wherein the polydimethylsiloxane exhibits a degree of polymerization of n=20-1000 and a kinetic viscosity of 20-2000 mm²/s

15. The highly emulsified silicone oil emulsion based medicament according to claim 12, comprising 0.01-35 w/w % hydrophobic emulsifier with a HLB-value of 1 to 7, and/or a hydrophilic emulsifier with a HLB-value of 7 to 14, and/or 0.01-35 w/w % of a mixture of one or more hydrophobic and hydrophilic emulsifiers with a HLB-value of 1 to 14.

16. The highly emulsified silicone oil emulsion based medicament according to claim 12, wherein the hydrophilic emulsifier is comprised of at least one of 25-35 w/w % sorbitan trioleate, 25-35 w/w % cetylstearyl alcohol, and 35-45 w/w % polysorbate 80.

17. The highly emulsified silicone oil emulsion based medicament according to claim 12, wherein the highly emulsified silicone oil emulsion contains 0.0001-1.0 w/w % chelate formers.

18. The highly emulsified silicone oil emulsion based medicament according to claim 12, wherein the highly emulsified silicone oil emulsion contains 0.0001-0.1 g/ml EDTA Na/Ca salt.

19. The highly emulsified silicone oil emulsion based medicament according to claim 12, wherein the highly emulsified silicone oil emulsion contains glycerol in a concentration of 0.01-50 w/w %.

20. The highly emulsified silicone oil emulsion based medicament according to claim 12, wherein the highly emulsified silicone oil emulsion is applied upon carrier material.

21. The highly emulsified silicone oil emulsion based medicament according to claim 20, wherein the carrier material is selected from the group cork, solid paraffins, waxes, resins, and woven textiles.

22. The highly emulsified silicone oil emulsion based medicament according to claim 12, wherein the highly emulsified silicone oil emulsion is combined with at least one of the following active substances:
antibiotic, corticoide, anti-Parkinson agent, agent against multiple sclerosis, neuro- or psychopharmaceuticals, cytostatics, immune stimulants, and analgesics.

23. The highly emulsified silicone oil emulsion based medicament according to claim 12, wherein the highly emulsified silicone oil emulsion is in solid, semi-solid, or liquid form.

24. The highly emulsified silicone oil emulsion based medicament according to claim 12, wherein the highly emulsified silicone oil emulsion contains one or more aides selected from the group cocoa butter, solidified plant oils, mixtures of mono, di- and triglyceride saturated fatty acids, waxes, cetyl stearyl alcohol, beeswax, glycerin monostearate, aluminum stearate, bentonite, celluloses, liquid triglycerides, paraffins, highly dispersed silica or diatomaceous earth, calcium carbonate, magnesium oxide, and antioxidants.

25. A highly emulsified silicone oil emulsion based medicament for the treatment of SIRS and/or sepsis in mammals, comprising
0.01-85 w/w % silicone,
0.01-90 w/w % of at least one emulsifier selected from
hydrophobic emulsifier with a HLB-level of 1 to 7,
hydrophilic emulsifier with a HLB-level of 7 to 14, and
mixture of one or more hydrophobic and hydrophilic emulsifiers with a HLB-level of 1 to 14, and p1 0.1-99 w/w % biocompatible saline solution
wherein said silicone and emulsifier are present in an amount sufficient to improve the ability of the mammal to recover from SIRS and sepsis.

26. A method for treatment of a mammal afflicted with SIRS and/or sepsis, said method comprising administering to a mammal afflicted with SIRS or sepsis at least one dose of a sufficient amount of a highly emulsified silicone oil emulsion based medicament to improve the ability of the mammal to recover from said affliction, wherein said highly emulsified silicone oil emulsion comprises:
0.01-85 w/w % silicone,
0.01-90 w/w % of at least one emulsifier selected from
hydrophobic emulsifier with a HLB-level of 1 to 7,
hydrophilic emulsifier with a HLB-level of 7 to 14, and
mixture of one or more hydrophobic and hydrophilic emulsifiers with a HLB-level of 1 to 14, and
0.1-99 w/w % biocompatible saline solution.

27. The method according to claim 26, wherein the silicone is a polydimethylsiloxane, and wherein the polydimethylsiloxane is emulsified.

28. The method according to claim 27, wherein the polydimethylsiloxane exhibits a degree of polymerization of n=20-1000and a kinetic viscosity of 20-2000 mm²/s

29. The method according to claim 26, wherein the medicament comprises 0.01-35 w/w % hydrophobic emulsifier with a HLB-value of 1 to 7, and/or a hydrophilic emulsifier with a HLB-value of 7 to 14,and/or 0.01-35 w/w % of a mixture of one or more hydrophobic and hydrophilic emulsifiers with a HLB-value of 1 to 14.

30. The method according to claim 26, wherein the hydrophilic emulsifier is comprised of at least one of 25-35 w/w % sorbitan trioleate, 25-35 w/w % cetylstearyl alcohol, and 35-45 w/w % polysorbate 80.

31. The method according to claim 26, wherein the mammal is human.
